(19) United States
(12) Patent Application Publication
(10) Pub. No.: US 2023/0305001 A1
(43) Pub. Date: Sep. 28, 2023

(54) ULTRA-SENSITIVE DIGITAL RAPID CHROMATOGRAPHIC ASSAY SYSTEM AND METHOD FOR ANALYTES DETECTION

(71) Applicant: SHANGHAI SKIN DISEASE HOSPITAL, Shanghai (CN)

(72) Inventors: Pengfei ZHANG, Shanghai (CN); Zhongjian CHEN, Shanghai (CN); Fei TAN, Shanghai (CN); Wannian YAN, Shanghai (CN); Lingzhi FAN, Shanghai (CN)

(73) Assignee: SHANGHAI SKIN DISEASE HOSPITAL, Shanghai (CN)

(21) Appl. No.: 18/022,994

(22) PCT Filed: Aug. 12, 2021
(86) PCT No.: PCT/CN2021/112311

§ 371 Date: Feb. 24, 2023

(30) Foreign Application Priority Data
Aug. 25, 2020 (CN) 202010860328.6

(51) Int. Cl. G01N 33/558; G01N 21/84; G01N 21/64

(52) U.S. Cl. CPC G01N 33/558; G01N 21/8483; G01N 21/6428

(57) ABSTRACT

An ultra-sensitive digital rapid chromatographic assay system includes a chromatography system, an optical imaging system, and an image processing system. The chromatography system is a lateral flow or vertical flow chromatography system. The detection area on a reaction membrane of the chromatography system is immobilized with capture biological ligands, the analytes to be detected is specifically enriched by means of the captured biological ligands, and the analytes enriched in the detection area is specifically recognized by the detection biological ligands labelled with tracer nanoparticles. The optical imaging system can visualize a single tracer nanoparticle specifically bound on the reaction membrane. The image processing system includes a detection area recognition module and a nanoparticle counting module, and the counted number of tracer nanoparticles specifically binding to the detection area and the concentration of the analytes to be detected are a proportional relationship.

### CROSS REFERENCE TO THE RELATED APPLICATIONS

This application is the national phase entry of International Application No. PCT/CN2021/112311, filed on Aug. 12, 2021, which is based upon and claims priority to Chinese Patent Application No. 202010860328.6, filed on Aug. 25, 2020, the entire contents of which are incorporated herein by reference.

### TECHNICAL FIELD

The present disclosure relates to a biological assay system, in particular relates to an ultra-sensitive rapid assay system for analytes detection, specifically relates to an ultra-sensitive digital rapid chromatographic assay system for analytes detection. In addition, the present invention still relates to a method for rapid detection of analytes by the ultra-sensitive digital chromatography system.

### BACKGROUND

A method of ultra-sensitive detection and analysis has broad application prospects in clinical disease detection, food safety, microbiological inspection and quarantine, veterinary medicine, etc. Digital detection and analysis technology refers to an absolute counting to specific reactions to improve the sensitivity of detection and analysis.

At present, common digital analysis techniques included digital PCR, digital ELISA, etc., the basic principle of which were to divide a bulk sample into millions of parts, distribute the target molecules to be detected in micro-reaction units and react in each reaction unit, after the reaction is completed, the number of micro-units with positive reaction is counted to achieve an ultra-sensitive detection of the target analytes. For example, a digital ELISA technology launched by Quanterix in the U.S. differs from the traditional immunoassays mainly in that it can capture a single molecule in femtoliter-sized microwells, allowing digital reading of the signal of a single magnetic bead, the sensitivity of this technology is 1000 times higher than that of the traditional ELISA method.

In addition, ultra-trace detection of target analytes can also be achieved by counting single nanoparticles that undergo specific binding reactions, as reported by Nongjian Tao, et al. (ACS Sens. 2020, 5, 4, 1126-1131), fixing antibodies on glass slides, combining with gold nanoparticles labeled antibody, observing single gold nanoparticles with a dark-field microscope and counting the specific bound gold nanoparticles to achieve super-sensitive detection of cardiac troponin. However, the above detection methods are cumbersome, time-consuming, and difficult to industrialize the detection system at low cost, so they are not suitable for developing on-site and rapid detection kits at point-of-care.

The Chinese invention patent application CN201811282815.8 disclosed an absolute quantitative method of low abundance protein based on digital immunoassay technology. In this method, after the immunoreaction of trapping magnetic beads, target antigen and detection particles, then the immune complex key detection particles were eluted, and the number of detection particles is analyzed with a microfluidic particle counting chip. However, the above digital immunoassay method also has the problems of complicated detection procedures and difficult standardization of reagents.

The Chinese invention patent application CN202010449078.7 disclosed a multi-spectral modulated portable quantitative detection device for immunochromatography test strip. In this invention, the optical detection module incidents the modulated light onto the test strips to be detected and guides the reflected light of C-line and T-line or fluorescence at a 45-degree receiving angle to the multi-spectral detection module. Since the above-mentioned detection method collects the macroscopic overall signal of the detected particles, it cannot detect and recognize the ultra-trace, especially the signal of a single detected particle, therefore the sensitivity of this analysis method is restricted.

### SUMMARY

A technical problem to be solved by the present invention is to provide a system for rapid detection of analytes by an ultra-sensitive digital chromatography, which overcomes the defects of cumbersome steps, long time consumption and difficult industrialization of detection systems in current digital assay methods, simplifies detection steps, shortens the detection time. The detection system is easy to standardize to achieve the ultra-sensitive and on-site rapid detection of the target analyte, and the analytical sensitivity of the existing chromatography detection technology is improved. Therefore, the present invention still provides a method for rapid detection of analytes by ultra-sensitive digital chromatography.

In order to solve the above technical problems, the technical solutions adopted in the present invention are:
an ultra-sensitive digital rapid chromatographic assay system for analytes detection, comprising: a chromatography reaction system, an optical imaging system, and an image processing system;

The chromatography reaction system is a lateral flow or vertical flow chromatographic reaction system, wherein the lateral flow chromatographic reaction system comprises a sample pad, a binding pad, a reaction membrane, and an absorbent pad; the vertical flow chromatographic reaction system comprises a reaction membrane, an absorbent paper, and an assembly cassette; a detection area on the reaction membrane of the chromatography reaction system is immobilized with capture biological ligands, and the analytes to be detected is specifically captured and enriched by means of the biological ligands, and the analytes enriched in the detection area is specifically recognized by the detection biological ligands labelled with tracer nanoparticles;

The optical imaging system is a fluorescence microscopy amplification or dark-field microscopy amplification optical system, which can visualize a single tracer nanoparticle specifically bound on the reaction membrane of the chromatography reaction system.

The image processing system comprises a detection area recognition module and a nanoparticle counting module, and the counted number of tracer nanoparticles specifically binds to the detection area and the concentration of the analytes to be detected being a proportional relationship.

As a preferred technical solution of the present invention, the chromatography reaction system is a lateral flow or vertical flow chromatographic reaction system, and the chromatography reaction time is finished in less than 20 minutes.

As a preferred technical solution of the present invention, the detection area of the chromatography reaction system is not only immobilized with specific capture biological ligands, but also immobilized with detection area marker particles. The detection area marker particles are fluorescent nanoparticles whose fluorescence emission wavelength is different from that of tracer particles or are particles of various shapes that can be distinguished under a microscopic imaging system.

Preferably, the particle diameter of the tracer nanoparticles is 10-500 nm with uniform particles diameter distribution, the tracer nanoparticles are fluorescent nanoparticles or plasmonic nanoparticles, the fluorescent nanoparticles are one or several combinations of time-resolved fluorescence, organic fluorescent dyes, fluorescent quantum dots, and aggregation-induced fluorescence, the plasmonic nanoparticles are one or more combinations of gold, platinum, silver, and palladium nanoparticles.

Preferably, the biological ligands are one or several combinations of antigen, antibody, nucleic acid aptamer, streptavidin, and biotin.

Preferably, the optical imaging system has a magnification of 100-1000 times and can visualize a single tracer nanoparticle.

Preferably, the detection area recognition module can recognize an image on the reaction membrane recorded by the optical imaging system, then recognize the detection area on the reaction membrane by means of the detection area marker particles.

Preferably, the nanoparticle counting module can count the number of nanoparticles specifically bound in the detection area.

In addition, the present invention still provides a method for rapid detection of analytes using the ultra-sensitive digital chromatography system, comprising the following steps:
Step 1, a certain volume of sample is dropwise added to a chromatography reaction system, after a chromatography reaction, microscopic images of the detection area on a reaction membrane of the chromatography reaction system are obtained using an optical imaging system, and tracer nanoparticles in the detection area are counted by an image processing system;
Step 2, the concentration of the analytes to be detected in the sample is calculated through a fitting relationship curve between the concentration of the calibrator and the number of tracer nanoparticles.

Compared with the prior art, the present invention has the following beneficial effects:

The present invention can significantly improve the sensitivity of the chromatography assay method through the combination of microscopic signal amplification and single nanoparticle counting. Besides immobilizing the capture ligand in the detection area, the marker nanoparticle is also immobilized to mark the detection area, so as to facilitate microscopic location of the detection area, the signal interference of non-specific binding between the reaction membrane and the marker particles was reduced. Through the amplification of the microscopic optical signal, the single particle counting of the tracer particles specifically bound to the detection area is achieved, and the relationship curve between the number of specific tracer particles in the detection area and the concentration of the marker to be detected was established. Compared with traditional naked eye interpretation or immunochromatographic analyzers based on bulk signal collection, the detection system and method can significantly improve the detection sensitivity of the fluorescence or colloidal gold immunochromatographic analysis method, and overcomes the defects of cumbersome steps, time-consuming and difficult standardization of the detection system of the existing digital assay method, simplifies the detection steps, shortens the detection time, and the detection system is easy to standardize, and has the advantages of chromatographic test paper of fast, convenient, low cost, and easy industrial production.

### BRIEF DESCRIPTION OF THE DRAWINGS

The present invention will be further described below in reference to the attached drawings.

FIG. 1 is a schematic diagram of the composition structure of the ultra-sensitive digital rapid chromatographic detection system of the present invention.

FIG. 2 is a schematic diagram comparing the detection results of the fluorescent membrane strips in a fluorescence imager and fluorescence microscope in Example 1 of the present invention, which is a comparison between the fluorescence imager photos of the fluorescence strips with different concentrations and the fluorescence microscope photos of the standard fluorescence strips with low concentration. In FIG. 2, the concentration of drawn membrane fluorescent particles in standard fluorescent membrane strips 1-8 is successively 1.2, 4.8, 19.2, 76.8, 307.2, 1228, 4915, and 19661 (hundred per centimeter).

FIG. 3 is a schematic diagram showing the comparison between the counting of microscopic particles of the standard fluorescent membrane strips and the detection results of the standard fluorescent membrane strips by the fluorescent immunoassay analyzer in Example 1 of the present invention.

FIG. 4 is a schematic structural diagram of the lateral chromatography reaction system in Example 2 of the present invention. In FIG. 4, 9-sample pad, 10-binding pad, 11-reaction membrane, 12-detection area, 13-quality control area, 14-absorbent pad, 15-PVC self-adhesive backboard.

FIG. 5 is the fluorescence microscope photos of the detection area of different detection concentrations of HIV p24 detected by quantum dot fluorescence lateral flow immunochromatography in Example 2 of the present invention.

FIG. 6 is a schematic diagram showing the comparison of detection results of different detection concentrations of HIV p24 detected by quantum dot fluorescence lateral immunochromatography in Example 2 of the present invention, respectively using fluorescence microscope counting and fluorescence immunochromatography analyzer.

FIG. 7 is a schematic structural diagram of the detection card of the vertical flow chromatography reaction system (dot-blot immunofiltration) in Example 3 of the present invention. In FIG. 7, 16-plastic cassette, 17-detection area, 18-reaction membrane, 19-absorbent pad.

FIG. 8 is a dark-field microscope photo of colloidal gold nanoparticles (40 nm) in Example 3 of the present invention.

### DETAILED DESCRIPTION OF THE EMBODIMENTS

The technical solutions in the examples of the present invention will be clearly and completely described below in reference to the attached drawings in the examples of the present invention. Obviously, the described examples are only a part of the examples of the present invention, not all of them. Based on the examples of the present invention, all other examples obtained by ordinary technical personnel in the field without making creative efforts fall within the protection scope of the present invention.

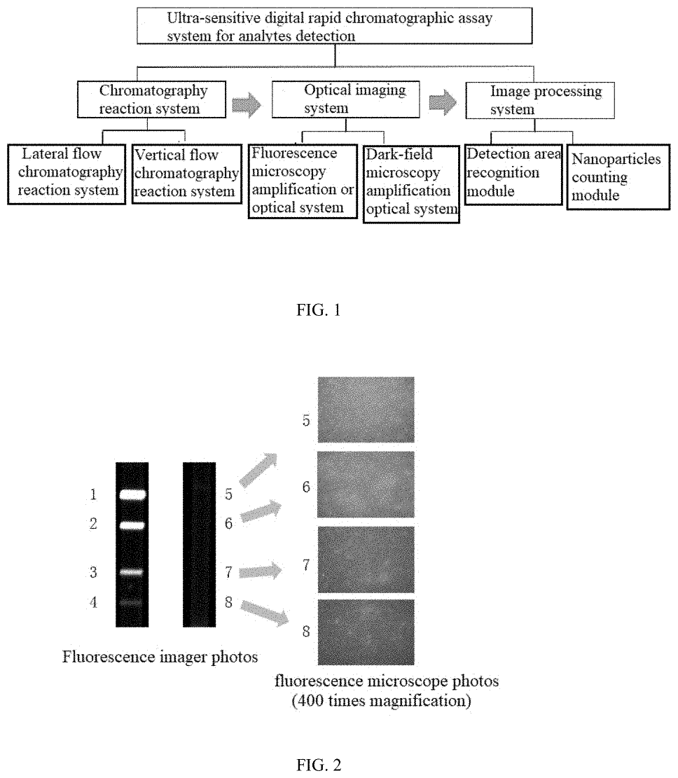

As shown in FIG. 1, the present invention an ultra-sensitive digital rapid chromatographic assay system for analytes detection, comprising: a chromatography reaction system, an optical imaging system, and an image processing system;

The chromatography reaction system is a lateral flow or vertical flow chromatographic reaction system, and the chromatography reaction time is less than 20 minutes, wherein the lateral flow chromatographic reaction system comprises a sample pad, a binding pad, a reaction membrane, and an absorbent pad; the vertical flow chromatographic reaction system comprises a reaction membrane, an absorbent paper, and an assembly cassette; a detection area on the reaction membrane of the chromatography reaction system is fixed with trapping biological ligands, and the enriched analytes to be detected is specifically captured by means of the biological ligands, and the analytes enriched in the detection area is specifically recognized by the detection biological ligands labelled with tracer nanoparticles; the particle diameter of the tracer nanoparticles is 10-500 nm with uniform particles diameter distribution, the tracer nanoparticles are fluorescent nanoparticles or plasmonic nanoparticles, the fluorescent nanoparticles are one or several combinations of time-resolved fluorescence, organic fluorescent dyes, fluorescent quantum dots, and aggregation-induced fluorescence, the plasmonic nanoparticles are one or more combinations of gold, platinum, silver, and palladium nanoparticles. The biological ligands are one or several combinations of antigen, antibody, nucleic acid aptamer, streptavidin, and biotin. The detection area of the chromatography reaction system is not only immobilized with specific capture biological ligands, but also immobilized with the detection area marker particles. The detection area marker particles are fluorescent nanoparticles whose fluorescence emission wavelength is different from that of tracer particles or are particles of various shapes that can be distinguished under a microscopic imaging system.

The optical imaging system is a fluorescence microscopy amplification or dark-field microscopy amplification optical system, which can distinguish a single tracer nanoparticle specifically bound on the reaction membrane of the chromatography reaction system; the optical imaging system has a magnification of 100-1000 times and can visualize a single tracer nanoparticle.

The image processing system comprises a detection area recognition module and a nanoparticle counting module, and the counted number of tracer nanoparticles specifically binds to the detection area and the concentration of the analytes to be detected being a proportional relationship. The detection area recognition module can recognize an image on the reaction membrane collected by the optical imaging system, then recognize the detection area on the reaction membrane by means of the detection area marker particles. The counting module specifically binding tracer nanoparticles can count the number of nanoparticles specifically binds in the detection area.

The present invention still provides a method for rapid detection of analytes using the above ultra-sensitive digital chromatography system, comprising the following steps:
Step 1, a certain volume of sample is dropwise added to a chromatography reaction system, after a chromatography reaction, a microscopic image of a detection area on a reaction membrane of the chromatography reaction system is obtained on an optical imaging system, and tracer nanoparticles in the detection area are counted by an image processing system;
Step 2, the concentration of the analytes to be detected in the sample is calculated through a fitting relationship curve between the concentration of the calibrator and the number of tracer nanoparticles.

### Example 1

Comparison of Sensitivity of Fluorescence Chromatography Analyzer and Fluorescence Microscope Photography Particles Counting

1. Preparation of Fluorescent Membrane Strips

After the quantum dot nanosphere microsphere solution was diluted 4 times sequentially, the nitrocellulose membrane (NC membrane) was fixed on the PVC self-adhesive backboard, and the membrane was drawn at a rate of 1 microliter per centimeter to obtain standard fluorescent bands with different fluorescence intensities.

2. Comparison of Detection Results of Standard Fluorescent Bands by Different Reading Methods

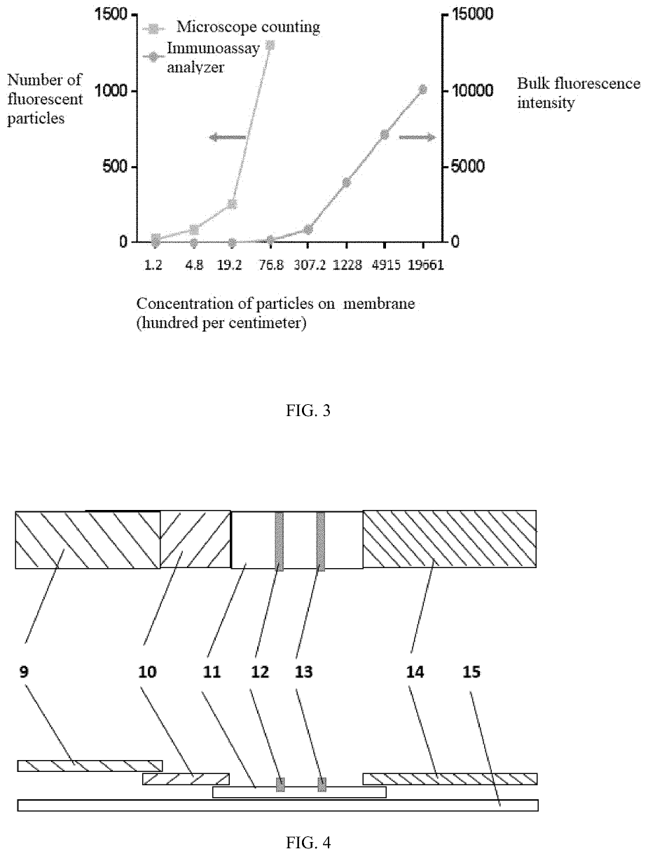

Fluorescence gel imager (Furi FR200 multifunctional imager), fluorescence immunochromatography analyzer (Suzhou Hemai FIC-H1) and fluorescence microscope (Olympus BX51) were respectively used to analyze the fluorescence signal of standard fluorescent membrane strips, the comparison of the detection results between the fluorescence imager and the fluorescence microscope photography is shown in FIG. 2, and the comparison between the detection results of the fluorescence immunochromatography analyzer and the fluorescence microscope counting is shown in FIG. 3, wherein the analysis sensitivity of the fluorescence imager and fluorescence immunochromatography analyzer is relatively close, and the detection sensitivity of fluorescent particles counting can be increased by about 100 times after taking pictures with a fluorescence microscope.

### Example 2

Ultra-Sensitive Detection of HIV p24 by Rapid Immunochromatographic Test Strips Based on Quantum Dot Fluorescence Labeling

1. Labeling of HIV p24 Antibody Labeled with Quantum Dot Nanospheres
1.1) 100 microliters of quantum dot nanospheres with carboxyl groups on the surface (emission wavelength 620 nm, red) was diluted to 300 microliters with pH 6.0 phosphate buffer, 0.3 mg of activator 1-(3-dimethylaminopropyl)-3-ethylcarbodiimide hydrochloride (EDC) was added, then placed in a rotary mixer, and reacted at room temperature for 0.5 hour.
1.2) After activation of the carboxy quantum dot microspheres, centrifuged at 12,000 rpm for 10 minutes, then after the supernatant was removed, the activated quantum dot nanospheres were dispersed in 300 microliters of phosphate buffer, and about 100 micrograms of HIV p24-labeled antibody was added, then placed in a rotary mixer, and reacted at room temperature for 1 hour.
1.3) After coupling reaction of the antibody and the quantum dot nanospheres, centrifuged at 12000 rpm for 10 minutes, the supernatant was removed, the antibody-quantum dot nanospheres were dispersed in 300 microliters of phosphate buffer, 10 mg of BSA was added, and placed in rotary mixer, and reacted at room temperature for 2 hours to obtain the HIV p24-quantum dot nanosphere conjugate.

2. Assembly of Digital Immunochromatographic Test Paper
2.1) Quantum dot nanospheres (emission wavelength 520 nm, green) were selected as the marker particles of the detection band, 100 microliter green quantum dot nanospheres were mixed with 10 mg of BSA, 0.3 mg of EDC was added, and placed in a rotary mixer, and reacted at room temperature for 2 hours to obtain tracer particles in the detection area.
2.2) CN 140 nitrocellulose membrane (NC membrane) of Sartorious was used as the chromatography reaction membrane, HIV p24 capture antibody at a concentration of 1 mg/ml and 1 microliter of maker particles were mixed as the detection area. Goat Anti-Mouse IgG polyclonal antibody at a concentration of 1 mg/ml was used as the quality control area; then, at a spray rate of 1 microliter per centimeter, the membrane is evenly drawn on the NC membrane to form a detection area and a quality control area separated by 4 millimeters.
2.3) The conjugate pad is made of hydrophilic glass fiber, and the HIV p24 labeled antibody-quantum dot nanosphere conjugate was dispersed in the treatment solution of the conjugate pad, and then evenly sprayed on the glass fiber membrane with a membrane sprayer and dried at 30° C. for use.
2.4) As shown in FIG. 4, the sample pad 9, the conjugate pad 10, the reaction membrane 11 and the absorbent pad 14 are stacked on the PVC self-adhesive backboard 15 as shown in FIG. 4, after assembling them into large plates, cut them into 4 mm wide test strips with a paper cutter.

2. Sample Testing and Interpretation of Results

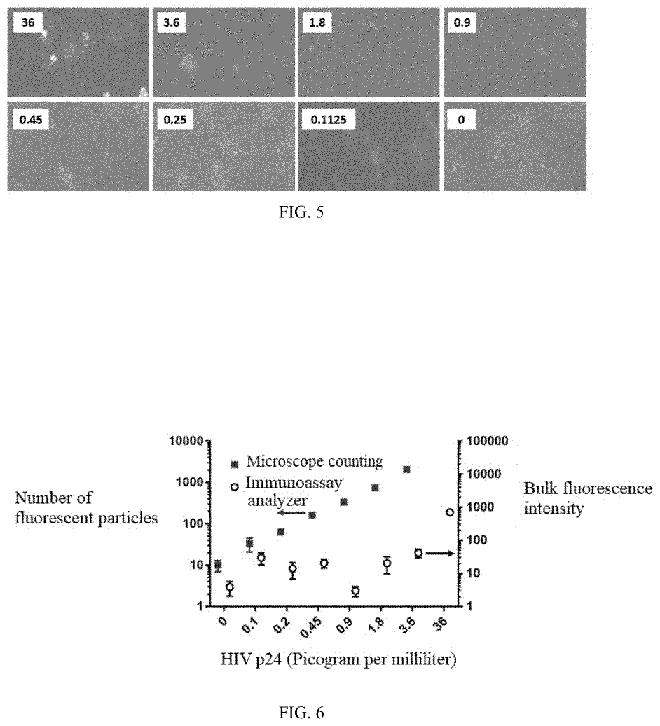

The calibrators or serum samples to be detected were diluted with buffer, and 100 microliters were added to the sample pad, after 10 minutes of reaction, the fluorescence signal intensity of the test strip and the quality control strip was detected by fluorescence immunochromatography; at the same time, a fluorescence microscope was used to take photos, and the fluorescence microscopic images of the detection strip were obtained through the micro-positioning of the detection area by the green fluorescence quantum dot, the fluorescence microscopic images were shown in FIG. 5, and the ImageJ software was used to count the red quantum dot nanospheres in the detection area. Wherein the comparison between the detection results of fluorescence immunochromatography analyzer and microscope counting was shown in FIG. 6. The concentration of HIV p24 in the sample was calculated by the fitting equation between a series of known concentrations of HIV p24 calibrators and the microscope counting of tracer particles.

### Example 3

Ultra-Sensitive Rapid Detection of C-Reactive Protein (CRP) by Colloidal Gold-Based Immunofiltration

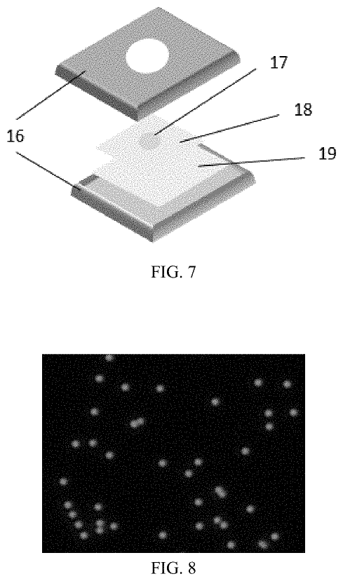

The commercial CRP colloidal gold immunofiltration detection kit was purchased from Shanghai Aopu Biological, and the detection structure was shown in FIG. 7, after diluting the calibrator solution with different gradients at multiple ratios, 100 microliters were added dropwise to the sample pad of the test strip, after 15 minutes of the chromatography reaction, the colloidal gold test plate was photographed under white light and grayscale analysis was carried out; photopgraphed with dark-field microscope, wherein the typical dark-field microscope photos of 40-nanometer colloidal gold particles are shown in FIG. 8, the colloidal gold particles in the detected spot unit area were counted, then the calibration curve was drawn, and the concentration of CRP in the sample to be detected was counted.

What is claimed is:

1. An ultra-sensitive digital rapid chromatographic assay system for analytes detection, comprising: a chromatography system, an optical imaging system, and an image processing system;
the chromatography system is a lateral flow chromatographic system or a vertical flow chromatographic system, wherein the lateral flow chromatographic system comprises a sample pad, a binding pad, a reaction membrane, and an absorbent pad; the vertical flow chromatographic system comprises the reaction membrane, an absorbent paper, and an assembly cassette; a detection area on the reaction membrane of the chromatography system is immobilized with capture biological ligands, and enriched analytes to be detected is specifically captured by the capture biological ligands, and the analytes enriched in the detection area is specifically recognized by detection biological ligands labelled with tracer nanoparticles; wherein the detection area of the chromatography system is immobilized with specific capture biological ligands and detection area marker particles;
the optical imaging system is a fluorescence microscopy amplification or dark-field microscopy amplification optical system, wherein the optical imaging system is configured to visualize a single tracer nanoparticle specifically bound on the reaction membrane of the chromatography system;
the image processing system comprises a recognition module of detection area and a counting module of specifically binding tracer nanoparticles, wherein a counted number of tracer nanoparticles specifically binding to the detection area and a concentration of the analytes to be detected are a proportional relationship; wherein the recognition module of detection area is configured to recognize an image on the reaction membrane collected by the optical imaging system, and then recognize the detection area on the reaction membrane by the detection area marker particles.

2. The ultra-sensitive digital rapid chromatographic assay system for analytes detection according to claim 1, wherein the chromatography system is the lateral flow chromatographic system or the vertical flow chromatographic system, and a testing time is less than 20 minutes.

3. The ultra-sensitive digital rapid chromatographic assay system for analytes detection according to claim 1, wherein the detection area marker particles are fluorescent nanoparticles or are particles of various shapes, wherein a fluorescence emission wavelength of the fluorescent nanoparticles is different from a fluorescence emission wavelength of tracer particles, and the particles of various shapes are able to be distinguished under a microscopic imaging system.

4. The ultra-sensitive digital rapid chromatographic assay system for analytes detection according to claim 1, wherein a particle diameter of the tracer nanoparticles is 10 nm-500 nm with uniform particles diameter distribution, and the tracer nanoparticles are fluorescent nanoparticles or plasmonic nanoparticles, wherein the fluorescent nanoparticles are one or a plurality of combinations of time-resolved fluorescent dyes, organic fluorescent dyes, fluorescent quantum dots, and aggregation-induced fluorescent luminogens, and the plasmonic nanoparticles are one or a plurality of combinations of gold, platinum, silver, and palladium nanoparticles.

5. The ultra-sensitive digital rapid chromatographic assay system for analytes detection according to claim 1, wherein the biological ligands are one or a plurality of combinations of antigen, antibody, nucleic acid aptamer, streptavidin, and biotin.

6. The ultra-sensitive digital rapid chromatographic assay system for analytes detection according to claim 1, wherein the optical imaging system has a magnification of 100 times-1000 times and is configured to distinguish the single tracer nanoparticle.

7. The ultra-sensitive digital rapid chromatographic assay system for analytes detection according to claim 1, wherein the counting module is configured to count the number of tracer nanoparticles specifically binding in the detection area.

8. A method for rapid detection of analytes using the ultra-sensitive digital rapid chromatographic assay system for analytes detection according to claim 1, comprising the following steps:
step 1, dropwise adding a certain volume of a sample to the chromatography system, after a testing action, obtaining a microscopic image of the detection area on the reaction membrane of the chromatography system by the optical imaging system, and counting the tracer nanoparticles in the detection area by the image processing system; and
step 2, calculating the concentration of the analytes to be detected in the sample through a fitting relationship curve between concentrations of calibrators and the counted numbers of the tracer nanoparticles.

9. The method according to claim 8, wherein in the ultra-sensitive digital rapid chromatographic assay system for analytes detection, the chromatography system is the lateral flow chromatographic system or the vertical flow chromatographic system, and a testing time is less than 20 minutes.

10. The method according to claim 8, wherein in the ultra-sensitive digital rapid chromatographic assay system for analytes detection, the detection area marker particles are fluorescent nanoparticles or are particles of various shapes, wherein a fluorescence emission wavelength of the fluorescent nanoparticles is different from a fluorescence emission wavelength of tracer particles, and the particles of various shapes are able to be distinguished under a microscopic imaging system.

11. The method according to claim 8, wherein in the ultra-sensitive digital rapid chromatographic assay system for analytes detection, a particle diameter of the tracer nanoparticles is 10 nm-500 nm with uniform particles diameter distribution, and the tracer nanoparticles are fluorescent nanoparticles or plasmonic nanoparticles, wherein the fluorescent nanoparticles are one or a plurality of combinations of time-resolved fluorescent dyes, organic fluorescent dyes, fluorescent quantum dots, and aggregation-induced fluorescent luminogens, and the plasmonic nanoparticles are one or a plurality of combinations of gold, platinum, silver, and palladium nanoparticles.

12. The method according to claim 8, wherein in the ultra-sensitive digital rapid chromatographic assay system for analytes detection, the biological ligands are one or a plurality of combinations of antigen, antibody, nucleic acid aptamer, streptavidin, and biotin.

13. The method according to claim 8, wherein in the ultra-sensitive digital rapid chromatographic assay system for analytes detection, the optical imaging system has a magnification of 100 times-1000 times and is configured to distinguish the single tracer nanoparticle.

14. The method according to claim 8, wherein in the ultra-sensitive digital rapid chromatographic assay system for analytes detection, the counting module is configured to count the number of tracer nanoparticles specifically binding in the detection area.
